(19) United States
(12) Patent Application Publication
(10) Pub. No.: US 2020/0352561 A1
(43) Pub. Date: Nov. 12, 2020

(54) SUTURE PASSER AND RELATED METHOD

(71) Applicants: Charles Getz, Newtown Square, PA (US); Vahid Entezari, Beachwood, OH (US)

(72) Inventors: Charles GETZ, Newtown Square, PA (US); Vahid ENTEZARI, Beachwood, OH (US)

(21) Appl. No.: 16/409,118

(22) Filed: May 10, 2019

(51) Int. Cl. A61B 17/04; A61F 2/08

(52) U.S. Cl. CPC A61B 17/0483; A61B 2017/00309; A61F 2/0811

(57) ABSTRACT

A suture passer for introducing a suture through two holes spaced at a fixed distance through a cortical portion of a patient's bone. The suture passer includes a body, a sheath and an introducer sheath. The body has a proximal end, a distal end and finger grips. A hollow portion extends through the body. The sheath is connected to the distal end of the body and is substantially hollow.

The sheath defines a longitudinal axis. The introducer sheath has a suture lumen extending therethrough generally parallel to the longitudinal axis. The introducer sheath is movably mounted within the hollow of the sheath. The introducer sheath includes a flexible tip. The introducer sheath is movable relative to the sheath between a retracted configuration and an extended configuration wherein the flexible tip has an approximate one hundred eighty degree (180°) turn.

### CROSS-REFERENCE TO RELATED APPLICATIONS

The present application claims the benefit of U.S. Provisional Patent Application No. 62/670,246, filed on May 11, 2018 and titled “Suture Passer and Related Method” the entire contents of which are incorporated herein by reference in its entirety.

### BACKGROUND OF THE INVENTION

Ligament repair and reconstruction is one of the most common procedures in orthopaedic surgery. In US alone, around three hundred seventy thousand (370,000) inpatient orthopaedic soft tissue procedures are performed annually for ligament repair and reconstruction. One of the major elements of these procedures is reattachment of native tendon/ligament or fixation of synthetic or allograft material to the patient's bone. Using suture anchors is the most common technique for reattaching the native tendon/ligament in this setting however, suture anchors are expensive. Some suture anchors are also prone to failure due to breakage, loosening and suture cutout, and may cause undesirable biological reactions.

Transosseous suture fixation (TOSF) was the standard method for open soft tissue repair without relying on suture anchors. This technique involves passing sutures between two holes on the patient's bone which can be technically challenging especially in an arthroscopic setting. Formation of the holes in the patient's bones at the appropriate location and passing a suture through the holes in a relatively simple manner are both challenges of the TOSF technique.

The present preferred invention addresses the shortcomings of prior art systems and methods.

### BRIEF SUMMARY OF THE INVENTION

The preferred present invention relates to a suture passer, preferably having a flexible tip. The preferred suture passer has a low profile with the flexible tip that allows reproducible passage of sutures between two drill holes on the bone. The preferred suture passer allows surgeons to perform the TOSF procedure, which often considered biomechanically superior to prior art procedures, without the need for expensive suture anchors.

Briefly stated, in one aspect, the preferred invention is directed to a suture passer for introducing a suture through two holes spaced at a fixed distance through a cortical portion of a patient's bone. The suture passer includes a body, a sheath and an introducer sheath. The body has a proximal end, a distal end and finger grips. A hollow portion extends through the body. The sheath is connected to the distal end of the body and is substantially hollow. The sheath defines a longitudinal axis. The introducer sheath has a suture lumen extending therethrough generally parallel to the longitudinal axis. The introducer sheath is movably mounted within the hollow of the sheath. The introducer sheath includes a flexible tip. The introducer sheath is movable relative to the sheath between a retracted configuration wherein the flexible tip is positioned within the sheath and extends substantially parallel to the longitudinal axis and an extended configuration wherein the flexible tip has an approximate one hundred eighty degree (180°) turn. A base of the flexible tip and a tip of the flexible tip define a first distance in the extended configuration. The first distance is substantially equal to the fixed distance.

In another aspect, the preferred invention is directed to a suture passer for introducing a suture through two holes spaced at a fixed distance through a cortical portion of a patient's bone. The suture passer includes a body having a proximal end, a distal end and finger grips. A hollow portion extends through the body. A sheath is connected to the distal end of the body and is substantially hollow. The sheath defines a longitudinal axis. An introducer sheath has a suture lumen extending therethrough generally parallel to the longitudinal axis. The introducer sheath is movably mounted within the hollow of the sheath. The introducer sheath includes a flexible tip. The introducer sheath is movable relative to the sheath between a retracted configuration wherein the flexible tip is positioned within the sheath and extends substantially parallel to the longitudinal axis and an extended configuration wherein the flexible tip has an approximate one hundred eighty degree (180°) turn. A base of the flexible tip and a tip of the flexible tip define a first distance in the extended configuration.

In yet another aspect, the preferred invention is directed to a suture passer for introducing a suture through two holes spaced at a fixed distance through a cortical portion of a patient's bone. The suture passer includes a body having a proximal end, a distal end and finger grips, a sheath connected to the distal end of the body and an introducer sheath having a suture lumen extending therethrough generally parallel to a longitudinal axis of the sheath. A hollow portion extends through the body. The sheath is substantially hollow. The introducer sheath is movably mounted within the hollow of the sheath. The introducer sheath includes a flexible tip that is comprised of a plurality of wedge-shaped segments. Each of the wedge-shaped segments includes a proximal angled surface and a distal angled surface. The introducer sheath is movable relative to the sheath between a retracted configuration wherein the flexible tip is positioned within the sheath and extends substantially parallel to the longitudinal axis and an extended configuration wherein the flexible tip has an approximate one hundred eighty degree)(180° turn. The proximal angled surfaces of the plurality of wedge-shaped segments and the distal angled surfaces are spaced from each other in the retracted position. The proximal angled surfaces and the distal angled surfaces are in facing engagement, respectively, in the extended configuration

### BRIEF DESCRIPTION OF THE SEVERAL VIEWS OF THE DRAWINGS

The foregoing summary, as well as the following detailed description of preferred embodiments of the suture passer, related parts of the suture passer and methods of the preferred invention, will be better understood when read in conjunction with the appended drawings. For the purposes of illustrating the preferred suture passers and related parts, there is shown in the drawings preferred embodiments. It should be understood, however, that the preferred invention is not limited to the precise arrangements and instrumentalities shown. In the drawings:

FIG. 1A is a top plan view of a suture passer in accordance with a first preferred embodiment of the present invention, wherein a flexible tip is in a retracted configuration;

FIG. 1B is a top plan view of the suture passer of FIG. 1A, wherein the flexible tip is in an extended configuration;

FIG. 2A is a magnified, top plan view of the flexible tip of the suture passer of FIG. 1A in the retracted configuration;

FIG. 2B is a magnified, top plan view of the flexible tip of the suture passer of FIG. 1A in the extended configuration;

FIG. 2C is a magnified, top plan view of the flexible tip of the suture passer of FIG. 1A in the extended configuration with a suture loaded in the flexible tip;

FIG. 3A is a top plan view of a suture passer in accordance with a second preferred embodiment of the present invention;

FIG. 3B is a side elevational view of the suture passer of FIG. 3A;

FIG. 3C is a cross-sectional view of a locking mechanism of the suture passer of FIG. 3A;

FIG. 4 is a top perspective view of the suture passer of FIG. 3A;

FIG. 4A is a magnified top perspective view of a thumb islet of the suture passer of FIG. 4, taken from within shape 4A of FIG. 4;

FIG. 4B is a magnified top perspective view of a retractable piece of the suture passer of FIG. 4, taken from within shape 4B of FIG. 4;

FIG. 4C is a magnified top perspective view of a flexible tip of the suture passer of FIG. 4, taken from within shape 4C of FIG. 4;

FIG. 4D is a magnified front perspective view of a central duct of the flexible tip of FIG. 4C, taken from within shape 4D of FIG. 4C

FIG. 5A is a magnified top perspective view of a flexible tip in accordance with a first alternative preferred embodiment, which may be utilized with either of the suture passers of FIGS. 1 and 3A;

FIG. 5B is a side elevational view of the flexible tip of FIG. 5A;

FIG. 6A is a magnified top perspective view of a flexible tip in accordance with a second alternative preferred embodiment, which may be utilized with either of the suture passers of FIGS. 1 and 3A; and

FIG. 6B is a side elevational view of the flexible tip of FIG. 6A.

### DETAILED DESCRIPTION OF THE INVENTION

Certain terminology is used in the following description for convenience only and is not limiting. Unless specifically set forth herein, the terms “a”, “an” and “the” are not limited to one element but instead should be read as meaning “at least one”. The words “right”, “left”, “lower” and “upper” designate directions in the drawings to which reference is made. The words “inwardly” or “distally” and “outwardly” or “proximally” refer to directions toward and away from, respectively, the patient's body, or the geometric center of the preferred suture passer and related parts thereof. The words, “anterior”, “posterior”, “superior,” “inferior”, “lateral” and related words and/or phrases designate preferred positions, directions and/or orientations in the human body or body parts to which reference is made and are not meant to be limiting. The terminology includes the above-listed words, derivatives thereof and words of similar import.

It should also be understood that the terms “about,” “approximately,” “generally,” “substantially” and like terms, used herein when referring to a dimension or characteristic of a component of the preferred embodiments of the invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

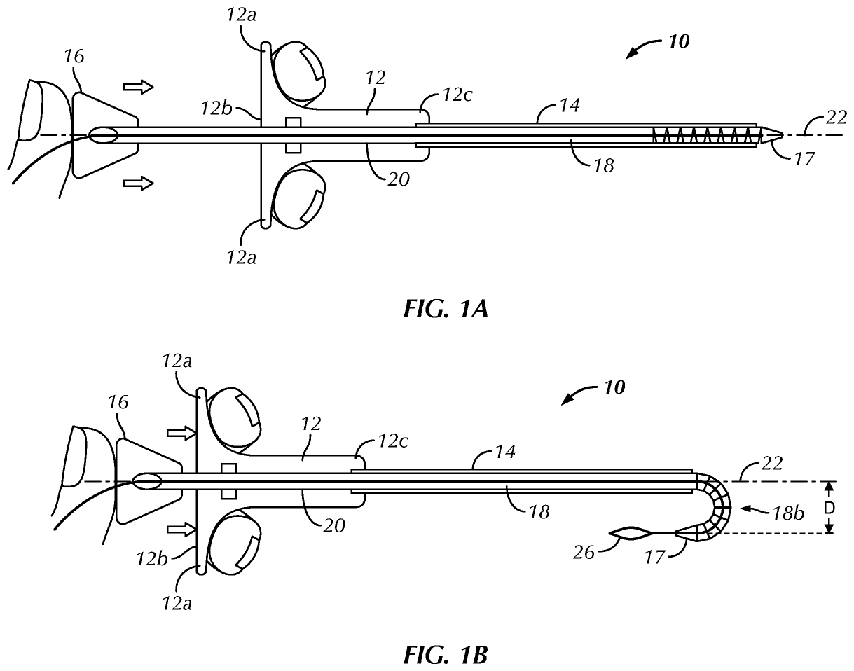

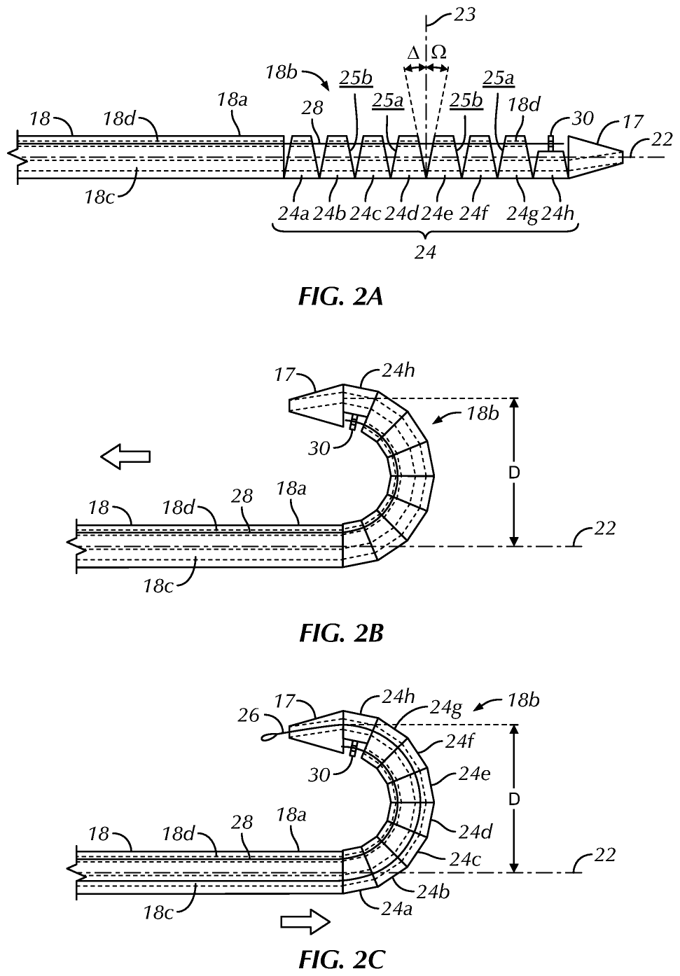

Referring to FIGS. 1A-2C, a suture passer, generally designated 10, in accordance with a first preferred embodiment includes a body 12, a hollow sheath 14 extending distally from the body 12, a plunger 16 movably mounted to a proximal side of the body 12 and an introducer shaft 18 mounted to the plunger 16 and movably positioned at least partially within the sheath 14. The body 12 preferably includes a pair of finger grips 12a that a surgeon or technician uses to grasp and hold the body 12 during use. The body 12 includes a hollow portion extending from a proximal end 12b to a distal end 12c that opens into the hollow sheath 14 in an assembled configuration. The introducer shaft 18 is preferably slidably mounted in the hollow portion of the body 12 and the hollow sheath 14 in the assembled configuration. The plunger 16 is fixed to a proximal end of the introducer shaft 18 for actuating the introducer shaft 18 from a biased, retracted configuration (FIG. 1A) to an extended configuration (FIG. 1B). The introducer shaft 18 is preferably biased to the retracted position by a spring 20 mounted between the body 12 and the introducer shaft 18, but is not so limited and may be otherwise biased relative to the body 12 to the retracted configuration, such as by alternate biasing mechanisms or methods, such as a spring-like material.

The introducer shaft 18 of the first preferred embodiment includes a proximal portion 18a and a flexible tip 18b positioned at a distal end of the introducer shaft 18. The proximal portion 18a is preferably substantially solid at its surface with a suture lumen 18c and an actuation lumen 18d extending therethrough along or generally parallel to a longitudinal axis 22. The flexible tip 18b also preferably includes the suture lumen 18c and the actuation lumen 18d, but is comprised of a series of wedge-shaped segments, generally designated by reference numeral 24, that allows an approximately one hundred eighty degree (180°) turn of the flexible tip 18b relative to the longitudinal axis 22 when the introducer shaft 18 is in the extended configuration (FIGS. 2B and 2C). In the first preferred embodiment, the series of wedge-shaped segments 24 is comprised of first, second, third, fourth, fifth, sixth, seventh and eighth segments 24a, 24b, 24c, 24d, 24e, 24f, 24g, 24h, but is not so limited and may be comprised of more or less wedge-shaped segments 24 or an alternative mechanism that permits turning or an arcuate flexing of the flexible tip 18b in the extended configuration. For example, the flexible tip 18b may be comprised of a relatively flexible material that may be manipulated by a user to turn or arc the flexible tip 18b as controlled by the user when moving from the retracted to the extended configuration or may be comprised of a shape-memory material that is held in a relatively straight configuration when in the retracted configuration and curves or arcs in the extended configuration.

In the first preferred embodiment, a suture 26 may be passed into and through the suture lumen 18c and out the end of the eighth segment 24h. An actuation element 28 is preferably positioned within the actuation lumen 18d in the assembled configuration and is fixed at its distal end to an anchor 30 on the eighth segment 24h in the first preferred embodiment. The actuation element 28 preferably biases the flexible tip 18b to the extended configuration or permits actuation of the flexible tip 18b to and between the relaxed configuration and the extended configuration. The first preferred suture passer 10 is not limited to inclusion of the actuation element 28 that is fixed to the anchor 30 on the eighth segment 24h to bias or actuate the flexible tip 18b and the flexible tip 18b may be otherwise designed and configured for moving between the relaxed and extended configurations, either automatically when the introducer sheath 18 moves relative to the sheath 14 or based on manipulation by the user. In the first preferred embodiment, the user is able to apply a tensile force on the actuation element 28 to urge the flexible tip 18b from the relaxed configuration to the extended configuration as the introducer sheath 18 moves from within the sheath 14 and to a position where the flexible tip 18b is exposed from the distal end of the sheath 14. The introducer sheath 18 is preferably moved from the relaxed configuration to the extended configuration and positions therebetween by engaging the finger grips 12a with the user's fingers and depressing the plunger 16 toward the body 12.

In the first preferred embodiment, the wedge-shaped segments 24, each have proximal and distal angled surfaces, which are designated generically by reference numbers 25a, 25b, that generally face each other in the retracted configuration and are in facing engagement in the extended configuration. Each of the distal surfaces 25b preferably extend at a first acute angle Δ relative to a plane 23 that is generally perpendicular to the longitudinal axis 22 and the proximal surfaces preferably extend at a second acute angle Ω relative to the plane 23. The first and second acute angles Δ, Ω are preferably, approximately ten to fifteen degrees (10-15°), but are not so limited and may be comprised of nearly any angle that results in the flexible tip 18b forming the approximately preferred one hundred eighty degree (180°) arc in the extended configuration. The first and second acute angles Δ, Ω are substantially the same in the first preferred embodiment, but are not so limited and may have differences between the different wedge-shaped segments 24a, 24b, 24c, 24d, 24e, 24f, 24g or may be otherwise designed and configured. In the first preferred embodiment, each of the wedge-shaped segments 24a, 24b, 24c, 24d, 24e, 24f, 24g has the proximal face 25a that defines the first acute angle Δ and the distal face 25b that defines the second acute angle Ω, wherein the first and second acute angles Δ, Ω are approximately twelve degrees (12°).

In use, two adjacent holes are drilled through the cortical portion of a target bone where attachment of a ligament or other soft tissue is desired. The distal end of the introducer sheath 14 is aligned with one of the holes with the suture 26 positioned in the suture lumen 18c and the user grasps the finger grips 12a and the plunger 16, preferably as shown in FIGS. 1A and 1B. The suture passer 10 is actuated by depressing the plunger 16 toward the body 12, thereby urging the introducer sheath 18 toward the distal end of the sheath 14 and the flexible tip 18b out of the sheath 14. The flexible tip 18b preferably has sufficient strength and rigidity to puncture and extend through the cancellous bone within the target bone. The user is then able to manipulate or the flexible tip 18b is biased to the extended configuration, such that the eighth segment 24h extends through and out of the second hole in the cortical bone by arcing around the cortical bone between the two holes. The suture 26 may be grasped to the user and utilized to secure the ligament or other soft tissue to the bone. The plunger 16 may then be released, resulting in the introducer sheath 18 moving back into the sheath 14. The first preferred suture passer 10 is then removed from the surgical site and an additional suture 26 may be loaded into the suture passer 10 for a subsequent procedure.

The first preferred suture passer 10 permits passing the suture 26 between the two drill holes in the cortex. The drill holes are preferably placed at a fixed distance relative to each other that may be defined by a drill guide in a kit that includes the preferred suture passer 10. In the extended configuration (FIGS. 1B, 2B and 2C) a base of the flexible tip 18b, generally at the longitudinal axis 22, and a tip 17 of the flexible tip 18b, preferably at an outlet of the suture lumen 18c, define a first distance D in the extended configuration. The first distance D is preferably substantially the same as the fixed distance such that the suture 26 is urged out of the hole during use. The first distance D substantially corresponds to the distance between the longitudinal axis 22 and the tip 17, which is attached to the eighth segment 24h, in the extended configuration such that the suture 26 extends out of the second drill hole in the patient's bone in the extended configuration. The tip 17 is preferably comprised, but not so limited, to a separate cone-shaped segment of the flexible tip 18b that facilitates passage of the tip 17 and flexible tip 18b through the patient's tissue, preferably soft tissue and cancellous bone, during use. The narrowing and cone-shape of the tip 17 facilitates a wedge-type movement of the tip 17 through the soft tissue and bone as the flexible tip 18b is urged through the soft tissue and bone between the two holes in the cortical bone or around an anatomical structure of the patient that the surgeon wants to avoid or surround to capture with the suture. The tip 17 is not limited to having the tip 17 or the tip 17 having the cone-shape and may function without the tip 17 or with a differently shaped tip 17, as is described in greater detail below. The tip 17 may be actuated by an actuation element 28, which is described in greater detail below, or may be biased toward the eighth segment 24h in the extended configuration to facilitate the arcuate, curved or turned shape of the flexible tip 18b in the extended configuration. The tip 17 is, however, preferably constructed with the cone-shape to allow the tip 17 to pierce through soft tissue and bone to facilitate maneuvering through a path created by transosseous drilling or to form its own path through the patient's soft tissue during use. The tip 17 includes the central duct 18c or an extension of the central duct 18c of the segments 24 to allow advancement of the suture 26 therethrough and may be connected to the actuation element 28, which may be comprised of a nitinol or shape-memory rod or a wire that facilitates manipulation of the flexible tip 18b between the extended and retracted configurations.

The first preferred suture passer 10 has a wide application in both open and arthroscopic orthopaedic procedures. The flexible tip 18b preferably has a substantially predictable one hundred eighty degree (180°) turn or arc in the extended configuration to facilitate predictability when drilling the holes and operating the suture passer 10. The suture passer 10 and its component parts are preferably constructed of a biocompatible resilient plastic or polymeric material that is able to take on the general size and shape of the suture passer 10 and its component parts, withstand the normal operating conditions of the suture passer 10 and its component parts and perform the preferred functions of the suture passer 10 and its components parts, as is described herein. The flexible tip 18b may be constructed of a biocompatible material that retains memory so the shape will return, preferably the relatively straight configuration of the retracted configuration or the arcuate shape in the extended configuration, when the stress is relaxed. The flexible tip 18b preferably has sufficient strength and stiffness to push through cancellous bone below the cortical bone during use so that the eighth segment 24h can be passed through the second drill hole or positioned at the base of the second drill hole to pass the end of the suture 26 out of the bone. The components of the suture passer 10 are not limited to biocompatible plastic or polymeric constructions and may be designed and constructed of metallic or other biocompatible material, such as stainless steel, that is able to take on the size and shape of the suture passer 10, withstand the normal operating conditions of the suture passer 10 and perform the preferred functions of the suture passer 10. The metallic suture passer 10 may also be configured for sterilization processing following use, such as by an autoclave sterilization process.

The first preferred suture anchor 10 allows “straight on” insertion of the sheath 14 into one of the holes or against one of the holes during use. The straight on insertion of the sheath 14 and suture passer 10 is relatively easy to use, both arthroscopically and during open procedures.

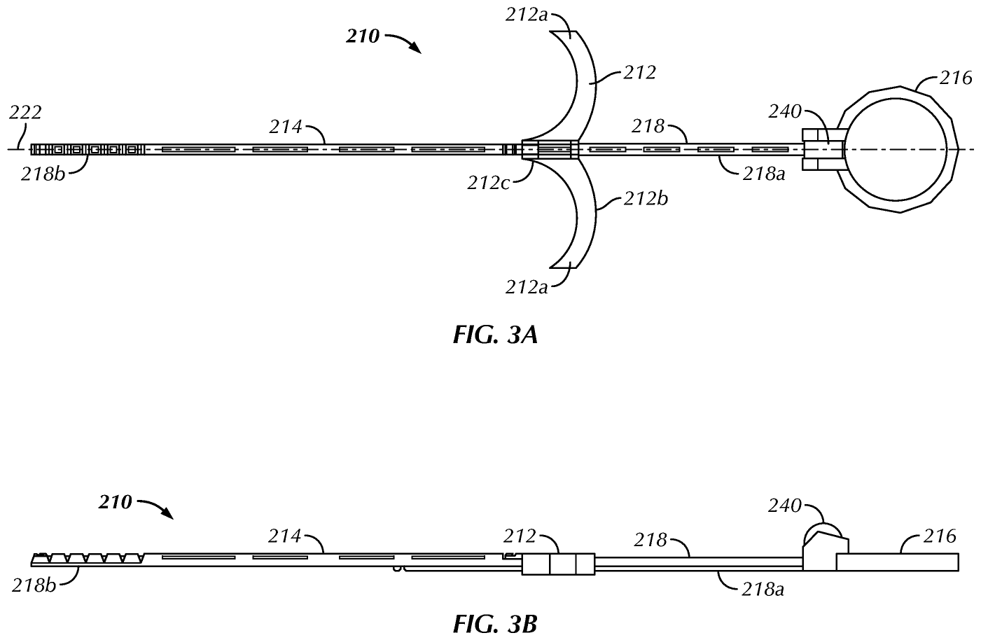

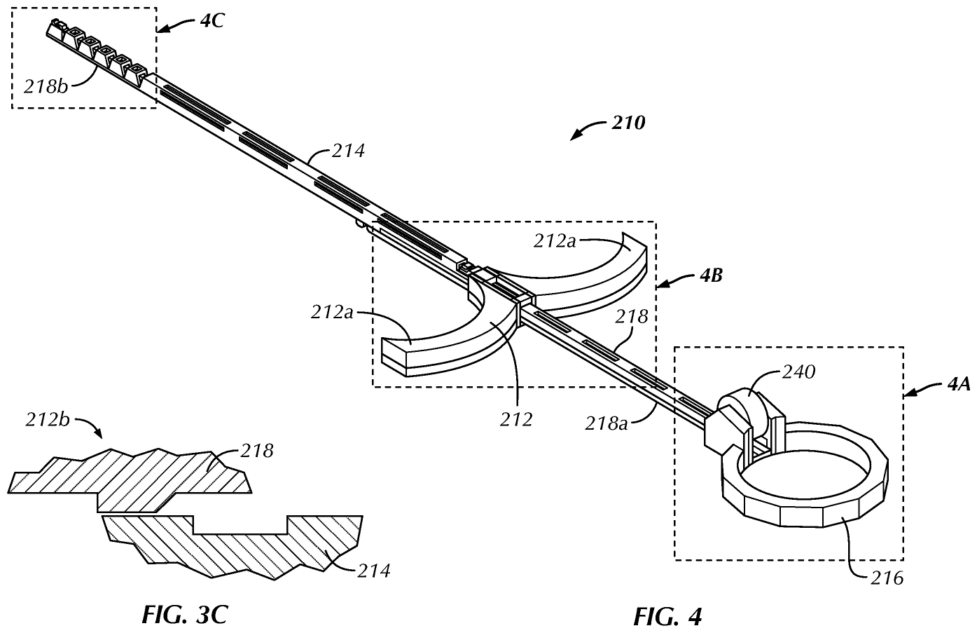

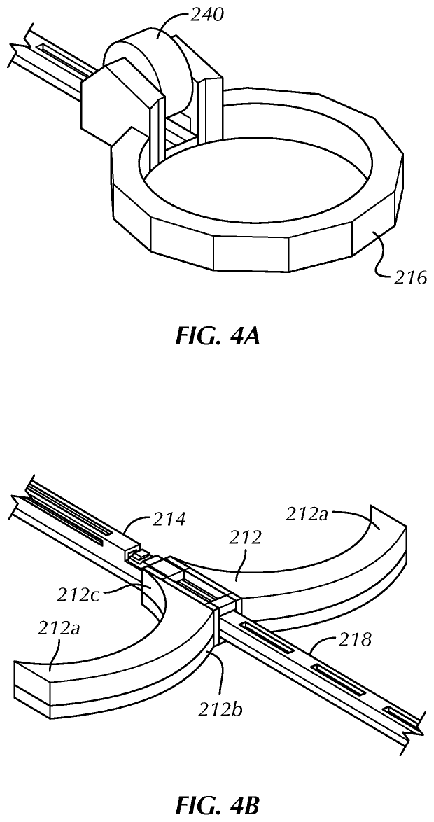

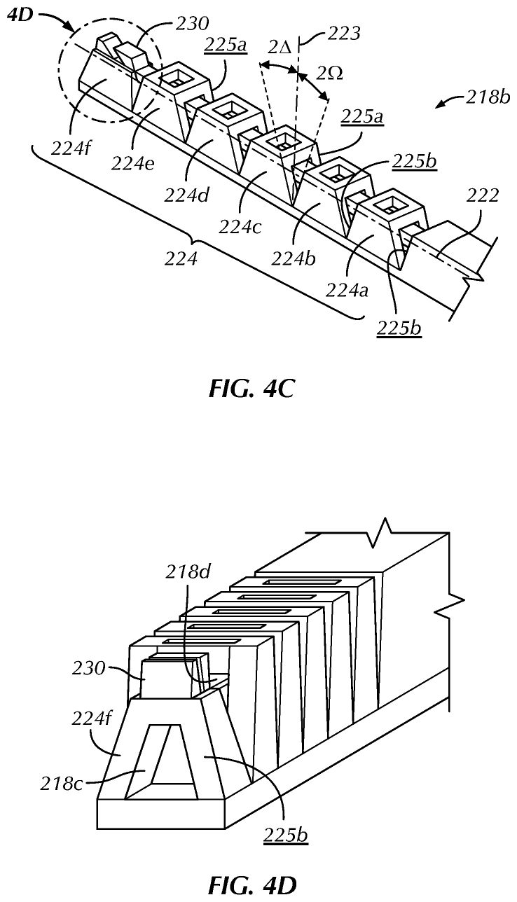

Referring to FIGS. 3A-4D, in a second preferred embodiment, a suture passer 210 has similar features to the suture passer 10 of the first preferred embodiment and like reference numerals are used to identify like features, with a “2” prefix used to distinguish the features of the second preferred embodiment from the features of the first preferred embodiment.

The second preferred suture passer 210 includes first, second, third, fourth, fifth and sixth segments 224a, 224b, 224c, 224d, 224e, 224f that comprise the flexible tip 218b with the anchor 230 positioned on the sixth segment 224f. The second preferred suture passer 210 is not limited to having six segments 224 and may include more or less wedge-shaped segments 224 or may be otherwise configured to direct the suture 26 from the first drill hole out of the second drill hole in the patient's bone. The flexible tip 218b is also constructed of a memory material that is able to “relax” when tension is removed to allow the flexible tip 218b to be removed from the bone with the flexible tip 218b automatically moving to the retracted position once tension is removed. Each of the wedge-shaped segments 224 includes the proximal and distal angled surfaces 225a, 225b that define the first and second acute angles 2Δ, 2Ω. In the second preferred embodiment, the first and second acute angles 2Δ, 2Ω are approximately each fifteen degrees (15°) to thereby define the approximately one hundred eighty degree (180°) arc of the flexible tip 218b in the extended configuration. The first and second acute angles 2Δ, 2Ω are not limited to this particular angle and the segments 224 are not limited to having the proximal and distal angled surfaces 225a, 225 and the flexible tip 218b may be otherwise designed and configured to facilitate positioning of the flexible tip 218b through the patient's bone and the two adjacent holes described above in the preferred procedure.

The flexible tip 218b and the proximal portion 218a preferably include the suture lumen or central duct 218c that allows passage of the suture and/or a central wire 26 and the retractable nitinol loop. The body 212 of the second preferred embodiment is preferably comprised of a retractable piece that allows the surgeon to retract the flexible tip 218b by holding the body 212 and plunger 216 like a syringe and squeezing the finger grips 212a with index and long fingers. The plunger 216 of the second preferred embodiment is preferably constructed of a thumb islet that allows the surgeon to push the device with the thumb. Pushing the plunger 216 with the thumb actuates a rolling wheel 240 to allow easy advancement of a central wire (not shown) that is positioned within the suture lumen or central duct 218c. The rolling wheel 240 allows the user to advance and retract the suture and/or central wire 26 by pressing down and turning the rolling wheel 240 in forward and backward directions. The introducer sheath 218 is preferably constructed of a nitinol loop. The second preferred suture passer 210 also preferably includes a locking mechanism 212b that engages a prominent portion of the introducer sheath 218 into a slot on the hollow sheath 214 that allows stabilization of the introducer sheath 218, particularly the flexible tip 218b, after the flexible tip 218b is turned or arced in the extended configuration. The flexible tip 218b is preferably locked in the extended position in order to facilitate the user's operation of the rolling button or wheel 240 to advance or retract the suture and/or central wire 26 while the flexible tip 218b is in the extended position. The locking mechanism 212b may likewise be disengaged by the user to release the introducer sheath 218 relative to the hollow sheath 214 such that the introducer sheath 218 may be positioned in the retracted position for removal of the flexible tip 218 and the suture passer 210 from the patient.

Referring to FIGS. 1-4D, the preferred suture passers 10, 210 are adaptable to a variety of fixation and suturing procedures, including various transosseous suture fixation procedures, wherein the flexible tip 18b, 218b facilitates passage of the suture 26 between two holes in the patient's bone. The preferred suture passers 10, 210 also facilitate passing the sutures 26 around anatomic structures of the patient and may also be utilized as a guide to establish portals for additional procedures during a surgery.

The preferred suture passers 10, 210 may be constructed of a series of individual elements that are assembled into the suture passers 10, 210 or may be integrally formed and constructed by three-dimensional (“3D”) printing in a single operation. The 3D printing process facilitates quick construction of an operational suture passer 10, 210 at any location, such as remote locations, where a 3D printer is available. The 3D printed version of the suture passer 10, 210 may also facilitate customization of the suture passer 10, 210 for specific procedures, operations or patients by modifying the size, shape or operability of the suture passer 10, 210 based on designer, user or surgeon preferences or for particularly portions of the procedure.

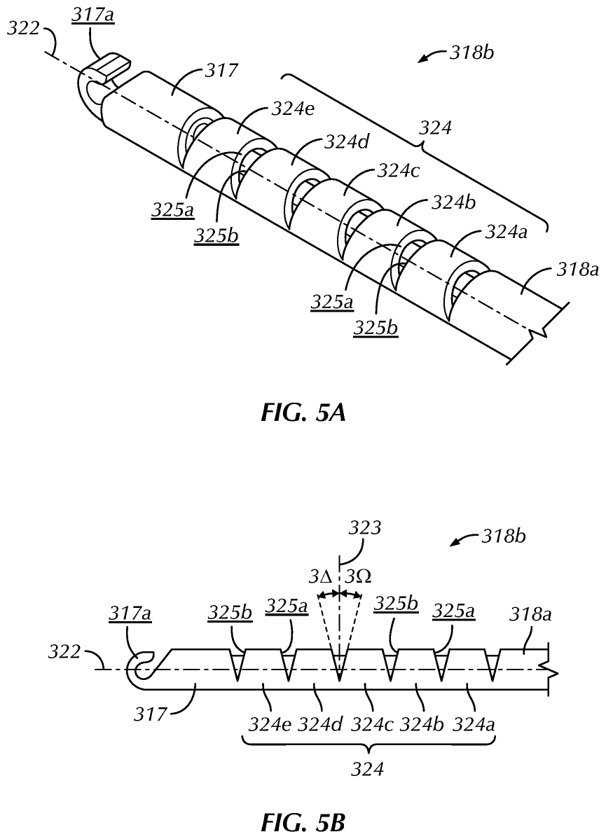

Referring to FIGS. 5A and 5B, in a first alternative preferred embodiment, a flexible tip 318b may be utilized with either of the first or second preferred suture passers 10, 210, has similar features to the flexible tips 18b, 218b of the first and second preferred embodiments and like reference numerals are used to identify like features, with a “3” prefix used to distinguish the features of the first alternative preferred embodiment of the flexible tip 318b from the features of the first and second preferred embodiments.

The first alternative preferred flexible tip 318b includes first, second, third, fourth and fifth segments 324a, 324b, 324c, 324d and 324e and the tip 317 that comprise the flexible tip 318b. The first alternative preferred flexible tip 318b is not limited to having five segments 324 and the tip 317 may include more or less wedge-shaped segments 324 or may be otherwise configured to direct the suture 26 from the first drill hole out of the second drill hole in the patient's bone. Each of the wedge-shaped segments 324 includes the proximal and distal angled surfaces 325a, 325b that define the first and second acute angles 3Δ, 3Ω. In the first alternative preferred embodiment, the first and second acute angles 3Δ, 3Ω are each approximately twelve to seventeen degrees (12-17°), most preferably approximately fifteen degrees (15°), to thereby define the approximately one hundred eighty degree (180°) arc of the flexible tip 318b in the extended configuration. The first and second acute angles 3Δ, 3Ω are not limited to this particular angle and the segments 324 are not limited to having the proximal and distal angled surfaces 325a, 325, as the flexible tip 318b may be otherwise designed and configured to facilitate positioning of the flexible tip 318b through the patient's bone and the two adjacent holes described above in the preferred procedure. The proximal portion 318a, the segments 324 and the tip 317 of the first alternative preferred embodiment preferably have a generally cylindrical shape with the longitudinal axis 322 extending coaxially through these components. The proximal portion 318a, the segments 324 and the tip 317 are not limited to having the generally cylindrical shape and may have other configurations and shapes, such as a boxy or other shape that is able to perform the preferred functions of these elements, withstand the normal operating conditions of the first alternative flexible tip 318b and take on the general size of the first alternative preferred flexible tip 318b. The tip 317 of the first alternative preferred embodiment includes a hook 317a at its distal end that may be utilized to retain and retract a suture 26 from the second hole in the patient's bone back through the first hole to secure the suture 26 to the patient's bone or to another structure. The hook 317a may also include a feature, such as a slot or groove (not show) to retain the suture 26 for functioning similar to the function described above, wherein the suture is passed from the first hole to the second hole during use. The hook 317a is preferably configured to allow pulling the suture from one side of an anatomical structure back to another side of the anatomical structure where the flexible tip 318b is inserted or initially positions, such as pulling the suture 26 from the second hole in the patient's bone back to and through the first hole in the patient's bone.

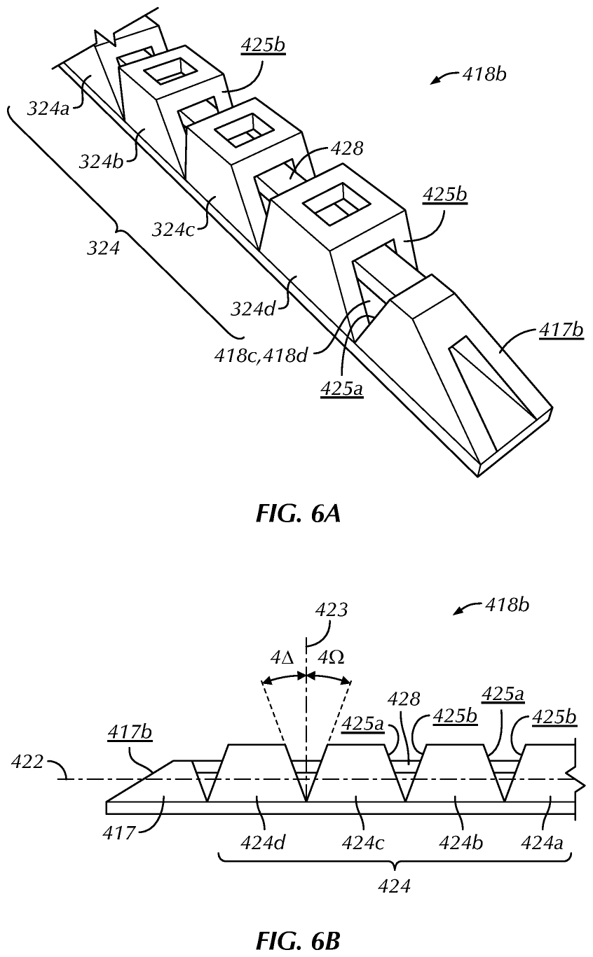

Referring to FIGS. 6A and 6B, in a second alternative preferred embodiment, a flexible tip 418b may be utilized with either of the first or second preferred suture passers 10, 210, has similar features to the flexible tips 18b, 218b, 318b of the first, second and first alternative preferred embodiments and like reference numerals are used to identify like features, with a “4” prefix used to distinguish the features of the second alternative preferred embodiment of the flexible tip 418b from the features of the first, second and first alternative preferred embodiments.

The second alternative preferred flexible tip 418b includes first, second, third and fourth segments 424a, 424b, 424c and 424d and the tip 417 that comprise the flexible tip 418b. The second alternative preferred flexible tip 418b is not limited to having four segments 424 and the tip 417 may include more or less wedge-shaped segments 424 or may be otherwise configured to direct the suture 26 from the first drill hole out of the second drill hole in the patient's bone. Each of the wedge-shaped segments 424 includes the proximal and distal angled surfaces 425a, 425b that define the first and second acute angles 4Δ, 4Ω. In the second alternative preferred embodiment, the first and second acute angles 4Δ, 4Ω are each approximately twenty to twenty-five degrees (20-25°), most preferably approximately twenty-two and one-half degrees (22½°), to thereby define the approximately one hundred eighty degree (180°) arc of the flexible tip 418b in the extended configuration. The first and second acute angles 4Δ, 4Ω are not limited to this particular angle and the segments 424 are not limited to having the proximal and distal angled surfaces 425a, 425, as the flexible tip 418b may be otherwise designed and configured to facilitate positioning of the flexible tip 418b through the patient's bone and the two adjacent holes described above in the preferred procedure. The proximal portion, the segments 424 and the tip 417 of the second alternative preferred embodiment preferably have a generally boxy or cube-type shape with the longitudinal axis 422 extending coaxially through these components. The segments 424 and the tip 417 are not limited to having the generally boxy or cube-type shape and may have other configurations and shapes, such as a cylindrical shape that is able to perform the preferred functions of these elements, withstand the normal operating conditions of the second alternative flexible tip 418b and take on the general size of the second alternative preferred flexible tip 418b. Any of the preferred flexible tips 18b, 218b, 318b, 418b may similarly have alternative shapes and configurations that are able to perform the preferred functions of the flexible tips 18b, 218b, 318b, 418b, withstand the operating conditions of the flexible tips 18b, 218b, 318b, 418b and take on the general size of the flexible tips 18b, 218b, 318b, 418b. The tip 417 of the second alternative preferred embodiment includes a distal sloped surface 417b at its distal end that facilitates moving the tip 417 through the patient's soft tissue and cancellous bone, similar to the cone-shaped tip 17 of the first preferred embodiment. The tip 417 also preferably includes a feature, such as a slot or groove (not show) to retain the suture 26 for functioning similar to the function described above, wherein the suture is passed from the first hole to the second hole during use. The actuation element 428 is preferably secured to a proximal side of the tip 417 to actuate the flexible tip 417b. The actuation element 428 of the second alternative preferred embodiment extends through the suture lumen and actuation lumen 418c, 418d, which are substantially combined in the second alternative preferred embodiment.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present disclosure.

We claim:

1. A suture passer for introducing a suture through two holes spaced at a fixed distance through a cortical portion of a patient's bone, the suture passer comprising:
a body having a proximal end, a distal end and finger grips, a hollow portion extending through the body;
a sheath connected to the distal end of the body and being substantially hollow, the sheath defining a longitudinal axis; and
an introducer sheath having a suture lumen extending therethrough generally parallel to the longitudinal axis, the introducer sheath movably mounted within the hollow of the sheath, the introducer sheath including a flexible tip, the introducer sheath movable relative to the sheath between a retracted configuration wherein the flexible tip is positioned within the sheath and extends substantially parallel to the longitudinal axis and an extended configuration wherein the flexible tip has an approximate one hundred eighty degree (180°) turn, a base of the flexible tip and a tip of the flexible tip define a first distance in the extended configuration.

2. The suture passer of claim 1, wherein the introducer sheath includes an actuation lumen extending substantially parallel to the longitudinal axis in the retracted position.

3. The suture passer of claim 1, further comprising:
a plunger fixed to a proximal end of the introducer sheath.

4. The suture passer of claim 1, wherein the flexible tip is comprised of at least six wedge-shaped segments.

5. The suture passer of claim 1, wherein at least one of the six wedge-shaped segments includes an anchor for fixing a distal end of an actuation element.

6. The suture passer of claim 1, wherein the first distance is approximately equal to the fixed distance.

7. The suture passer of claim 1, wherein the flexible tip includes a first wedge-shaped segment.

8. The suture passer of claim 7, wherein the first wedge-shaped segment includes a first proximal angled surface and a first distal angled surface, a plane defined generally perpendicular relative to the longitudinal axis, the first proximal angled surface defining a first acute angle relative to the plane and the first distal angled surface defining a second acute angle relative to the plane.

9. The suture passer of claim 8, wherein the first acute angle is approximately ten to fifteen degrees (10-15°).

10. The suture passer of claim 1, wherein the flexible tip is comprised of a plurality of wedge-shaped segment.

11. The suture passer of claim 10, wherein each of the wedge-shaped segments includes a proximal angled surface and a distal angled surface.

12. The suture passer of claim 11, wherein each of the proximal angled surfaces define a first acute angle relative to a plane defined substantially perpendicular to the longitudinal axis and each of the distal angled surfaces define a second acute angle relative to the plane.

13. The suture passer of claim 12, wherein the first acute angle is substantially equal to the second acute angle.

14. The suture passer of claim 12, wherein the first and second acute angle are approximately ten to fifteen degrees (10-15°).

15. A suture passer for introducing a suture through two holes spaced at a fixed distance through a cortical portion of a patient's bone, the suture passer comprising:
a body having a proximal end, a distal end and finger grips, a hollow portion extending through the body;
a sheath connected to the distal end of the body and being substantially hollow, the sheath defining a longitudinal axis; and
an introducer sheath having a suture lumen extending therethrough generally parallel to the longitudinal axis, the introducer sheath movably mounted within the hollow of the sheath, the introducer sheath including a flexible tip, The flexible tip comprised of a plurality of wedge-shaped segments, each of the wedge-shaped segments including a proximal angled surface and a distal angled surface, the introducer sheath movable relative to the sheath between a retracted configuration wherein the flexible tip is positioned within the sheath and extends substantially parallel to the longitudinal axis and an extended configuration wherein the flexible tip has an approximate one hundred eighty degree (180°) turn, the proximal angled surfaces of the plurality of wedge-shaped segments and the distal angled surfaces spaced from each other in the retracted position and in facing engagement, respectively, in the extended configuration.

16. The suture passer of claim 15, wherein the plurality of wedge-shaped segments includes eight wedge-shaped segments.

17. The suture passer of claim 15, wherein the plurality of wedge-shaped segments includes six wedge-shaped segments.

18. The suture passer of claim 15, wherein each of the proximal angled surfaces define a first acute angle relative to a plane defined perpendicular to the longitudinal axis and each of the first acute angles are substantially the same.

19. The suture passer of claim 15, wherein each of the distal angled surfaces define a second acute angle relative to a plane defined perpendicular to the longitudinal axis and each of the second acute angles are substantially the same.

20. The suture passer of claim 15, wherein the introducer sheath includes an actuation lumen extending therethrough substantially parallel to the longitudinal axis.
